(19) United States
(12) Patent Application Publication
(10) Pub. No.: US 2013/0041250 A1
(43) Pub. Date: Feb. 14, 2013

(54) METHODS AND APPARATUS FOR LOCATING ARTERIES AND VEINS USING ULTRASOUND

(71) Applicants: Laurent Pelissier, North Vancouver (CA); Joseph Bjorklund, Menomonee Falls, WI (US)

(72) Inventors: Laurent Pelissier, North Vancouver (CA); Joseph Bjorklund, Menomonee Falls, WI (US)

(73) Assignee: Ultrasonix Medical Corporation

(21) Appl. No.: 13/570,136

(22) Filed: Aug. 8, 2012

Related U.S. Application Data
(60) Provisional application No. 61/521,671, filed on Aug. 9, 2011.

(51) Int. Cl. A61B 8/06; A61B 8/13

(52) U.S. Cl. 600/424

(57) ABSTRACT

A method for targeting a blood vessel comprises acquiring ultrasound data, automatically determining a target sample volume, acquiring Doppler ultrasound data, and communicating an indicator of flow characteristics in the target sample volume. The ultrasound data is for a plane within a body. The target sample volume is determined at an intersection of a trajectory of an insertable instrument with the plane. The Doppler ultrasound data is acquired for at least the target sample volume at the intersection. The indicator of flow characteristics is based on the Doppler ultrasound data. An associated apparatus is also described.

### CROSS REFERENCE TO RELATED APPLICATIONS

This application claims priority from U.S. Patent Application No. 61/521,671 filed on 9 Aug. 2011 and entitled METHODS AND APPARATUS FOR LOCATING ARTERIES AND VEINS USING ULTRASOUND. For the purposes of the United States, this application claims the benefit under 35 U.S.C. §119 of U.S. Patent Application No. 61/521671 filed on 9 Aug. 2011 and entitled METHODS AND APPARATUS FOR LOCATING ARTERIES AND VEINS USING ULTRASOUND which is hereby incorporated herein by reference for all purposes.

### TECHNICAL FIELD

The application relates to medical ultrasonography. Embodiments provide methods and apparatus for assisting a user in locating arteries or veins in a body.

### BACKGROUND

Some medical procedures involve insertion of objects into blood vessels. For example, some medical procedures involve the insertion of the tips of needles into blood vessels, such as for drawing blood (e.g., for diagnostic purposes) or introducing fluids (e.g., for therapeutic purposes).

Ultrasound imaging is used to obtain images of structures in the bodies of humans and animals. In ultrasound imaging, ultrasound pulses are transmitted into a body, and reflected off of structures in the body (e.g., interfaces where there is a density change in the body). Reflected ultrasound pulses (echos) are detected at a transducer. Timing and strength information for reflected pulses is used to construct images. Ultrasound images may be used in guiding insertion of objects into a body during medical procedures.

In some medical procedures, it is important that the blood vessel in which an object is inserted is one or the other of a vein and an artery. Veins and arteries may be located close together, and their shapes may be somewhat similar. As a result, persons performing medical procedures may have difficulty distinguishing veins from arteries depicted in ultrasound images.

There is a desire for methods and apparatus that help persons performing medical procedures to locate arteries or veins in a body.

The foregoing examples of the related art and limitations related thereto are intended to be illustrative and not exclusive. Other limitations of the related art will become apparent to those of skill in the art upon a reading of the specification and a study of the drawings.

### BRIEF DESCRIPTION OF DRAWINGS

In drawings that illustrate non-limiting example embodiments:

FIG. 1 is a schematic diagram of an ultrasound system according to an example embodiment.

FIG. 2 is a schematic diagram of an ultrasound system according to an example embodiment.

FIG. 3 is a schematic diagram of an Pulse-Wave Doppler ultrasound system used to measure blood velocity in a blood vessel.

FIG. 4 is a flowchart of a method according to an example embodiment for communicating an indicator of blood flow characteristics at an intersection of a trajectory of an insertable instrument and a plane in a body.

FIG. 5 is a flowchart of a method according to an example embodiment for fixing the location of a target sample volume.

FIG. 6 is a flowchart of a method according to an example embodiment for determining whether blood flow characteristics at a target sample volume is consistent an artery or a vein.

FIG. 7 is a flowchart of a method according to an example embodiment for determining whether a flow characteristic value for a sample volume is consistent with an artery or a vein.

FIG. 8 is a diagram illustrating typical ranges of blood flow velocities for arteries, veins and anatomical structures that are neither arteries nor veins.

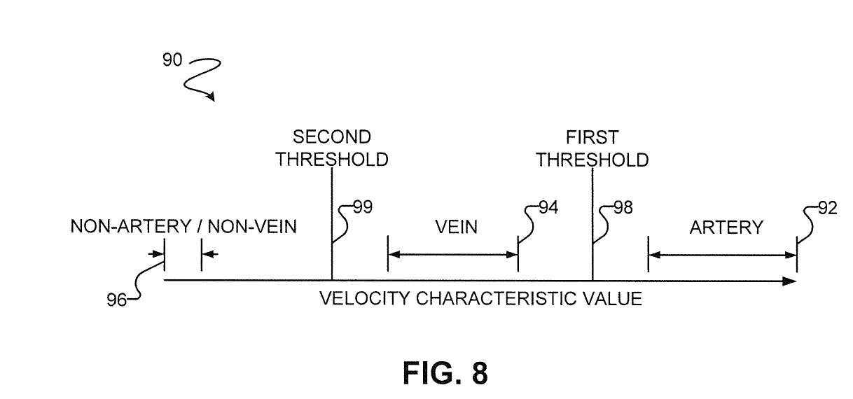

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

Some embodiments provide ultrasound systems which include needles or probes that can be inserted into a living subject along a trajectory. The ultrasound systems may be configured to indicate the trajectory on a display which also shows an ultrasound image of tissues in the subject. The needles or probes may be guided by a guide to intersect a plane of the image or free-hand. The trajectory may be determined by way of a position-sensing system and/or known as a result of the alignment of a guide. The ultrasound system may be configured to automatically perform Doppler ultrasound at a location corresponding to the intersection of the trajectory with the ultrasound image. Doppler ultrasound data can then be processed to determine whether the Doppler ultrasound data is characteristic of an artery (pulsatile blood flow), a vein (less pulsatile blood flow), or neither (blood flow below a threshold). An indicator may be operated to indicate to a user when the trajectory will intersect an artery (or a vein, as desired). A relatively inexperienced user may operate such a system to locate a blood vessel of interest, verify the blood vessel is an artery (or vein, as desired), and then insert the needle or probe into the blood vessel.

The following description describes components that may be used in such systems and describes how those components may be integrated.

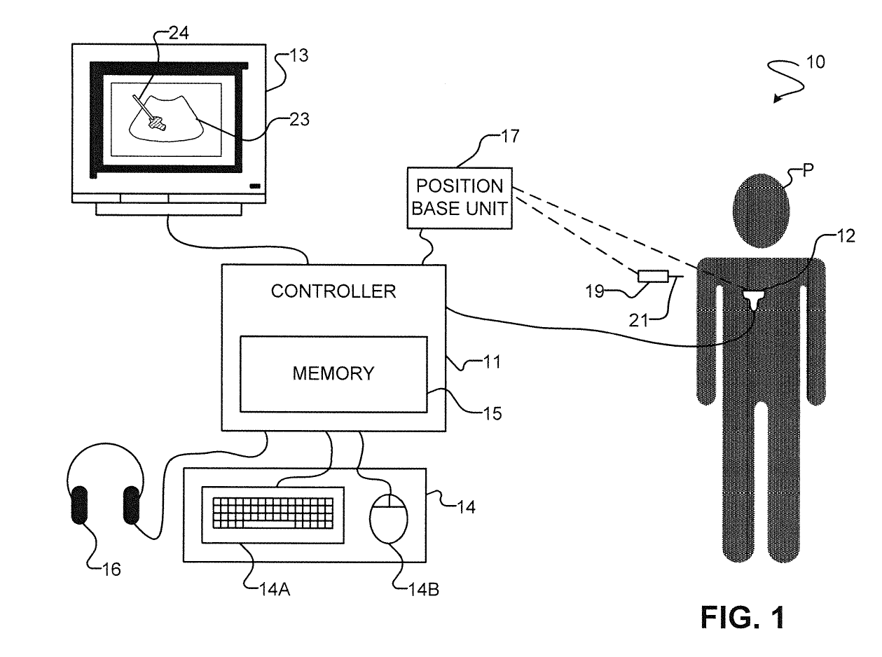

FIG. 1 shows an ultrasound system 10 according to an example embodiment. System 10 comprises a controller 11 connected to an ultrasound probe 12, a display 13, a user input device 14, and an audio monitor, namely headphones 16.

Ultrasound probe 12 emits ultrasound pulses into the body of patient P. Ultrasound pulses emitted by probe 12 are reflected off of structures in the body of patient P. Probe 12 receives reflected ultrasound pulses that return in its direction. Controller 11 may be configured to control aspects of the operation of ultrasound probe 12. For example, controller 11 may control the transmission of pulses from ultrasound probe 12 and/or the gating of samples of reflected pulses received at ultrasound probe 12.

Controller 11 may comprise one or more central processing units (CPUs), one or more microprocessors, one or more field programmable gate arrays (FPGAs), application specific integrated circuits, logic circuits, or any combination thereof, or any other suitable processing unit(s) comprising hardware and/or software capable of functioning as described herein.

Controller 11 comprises a memory 15. In some embodiments, memory 15 is external to controller 11. In other embodiments, memory 15 is part of controller 11. Controller 11 may be configured to store data representative of signals acquired by probe 12 in memory 15. Controller 11 processes ultrasound data acquired from ultrasound transducer 12. Controller 11 may receive ultrasound data for processing from memory 15 and/or probe 12. Controller 11 may be configured to process ultrasound data to generate B-mode or other images derived from the ultrasound data. Controller 11 may be configured to store B-mode image data in memory 15 and/or to display B-mode images on display 13.

Either or both of controller 11 and probe 12 may be provided by an ultrasound machine that is commercially available. Controller 11 and probe 12 may be of any known or future-developed type capable of acquiring ultrasound data, including ultrasound image data and/or Doppler ultrasound data.

Input device 14 provides user input to controller 11. In the illustrated embodiment, input device 14 comprises keyboard 14A and computer mouse 14B. Input device 14 may comprise other user interfaces. In some embodiments, display 13 comprises a touch screen, which may form part of input device 14. In some embodiments, ultrasound system 10 is provided as a hand-holdable unit which incorporates a probe, display, user interface, or controller.

A user may use input device 14 to control aspects of the operation of controller 11. Input device 14 may provide controls for manipulating the images generated by controller 11. For example, a user may interact with input device 14 to control parameters affecting acquisition of an ultrasound image, such as, for example, the gating of samples received at ultrasound probe 12 and thereby change the image displayed by controller 11 on display 13.

Control may be provided for other aspects of controller 11, such as steering the beam of ultrasound emitted by ultrasound probe 12. Transmit and/or receive focusing and steering of ultrasound pulses may be achieved, for example, through dynamic delay line control of the transmit and receive elements of probe 12.

System 10 also includes a position base unit 17 and an insertable instrument 19. A position-sensing system permits the spatial positions and orientations of insertable instrument 19 and probe 12 to be monitored in real time. In the illustrated embodiment, insertable instrument 19 and probe 12 are each associated with sensors (not shown), and position base unit 17 is operable to determine the locations of the sensors in space. Position base unit 17 and/or controller 11 is configured to determine the positions and orientations of probe 12 and insertable instrument 19 (and/or portions thereof) relative to one another based on the sensed locations of the sensors in space. Position base unit 17 and the sensors associated with probe 12 and instrument 19 may be provided by a known position sensor system, such as, for example, the position sensor systems described in co-owned U.S. patent application Ser. No. 12/703,706 entitled ULTRASOUND SYSTEMS INCORPORATING POSITION SENSORS AND ASSOCIATED METHODS and co-owned U.S. patent application Ser. No. 12/775,403, entitled FREEHAND ULTRASOUND IMAGING SYSTEMS AND METHODS FOR GUIDING ELONGATE INSTRUMENTS, which are hereby incorporated herein by reference in their entirety.

Having knowledge of the location of instrument 19 relative to anatomy imaged by probe 12 can permit the generation and display of images and other feedback that helps a user to visualize the relative locations of instrument 19 and anatomical structures within a patient P. For example, controller 11 may be configured to combine ultrasound data acquired by probe 12 and position information for probe 12 and instrument 19 to generate an enhanced ultrasound image 23. In ultrasound image 23, instrument 19 is represented in image 23 by a computer-generated line 24 or other indicia that shows the position of instrument 19 relative to anatomical structures depicted in image 23. Ultrasound image 23 may depict one or more anatomical structures, such as blood vessels, for example, into which it is desired to place part of instrument 19 (e.g., a needle 21 of instrument 19), and graphical elements (e.g., line 24) indicative of the position and/or trajectory of instrument 19 (or portions thereof, such as needle 21) relative to the anatomical structures may be displayed as part of enhanced ultrasound.

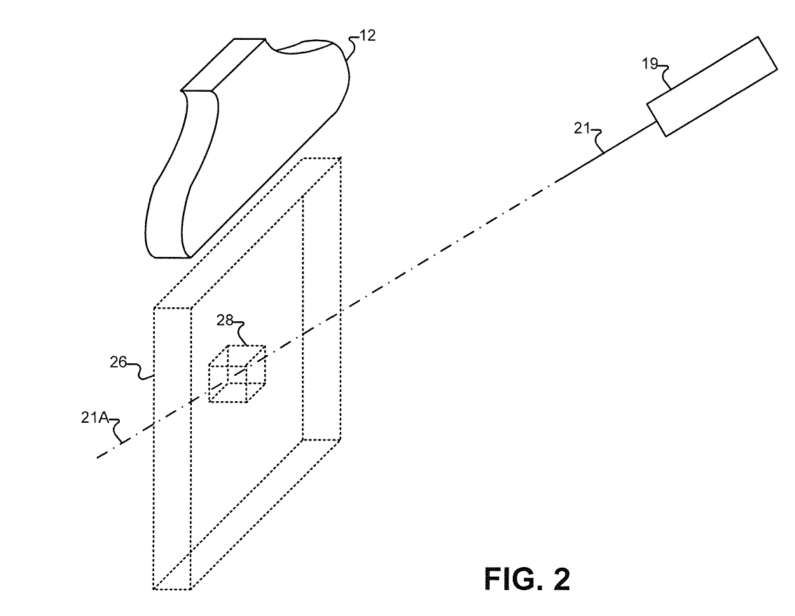

Enhanced images, such as image 23, may be generated using any suitable technique, such as, for example, those disclosed in US Patent Application publication no. 2010/298705, entitled FREEHAND ULTRASOUND IMAGING SYSTEMS AND METHODS FOR GUIDING FINE ELONGATE INSTRUMENTS, which is hereby incorporated herein by reference for all purposes.

Where system 10 is used in a medical procedure requiring insertion of needle 21 into a vein or artery, the person(s) performing the procedure may attempt to position probe 12 so that ultrasound system 10 images the vein or artery into which it is desired to insert needle 21 and position needle 21 so that its trajectory intersects the portion of the vein or artery shown in image 23. Where this is done, needle 21 will be positioned such that its trajectory intersects the region of patient P depicted in image 23. FIG. 2 shows an example of this. FIG. 2 is a perspective view of probe 12 positioned to image region 26 of the body of patient P. Probe 12 receives echos from anatomical structures in region 26 and generates corresponding ultrasound data. Controller 11 (not shown in FIG. 2) may then generate an ultrasound image of the anatomical structures in region 26 based on this data. Needle 21 and its trajectory 21A intersect region 26 at volume 28. In the illustrated embodiment, region 26 comprises a plane within the body of patient P.

It may occur that region 26 includes other anatomical structures that in image 23 appear similar to the target artery or vein. The target artery or vein may be distinguishable from these other anatomical structures by the fact that blood flows in arteries and veins, but does not flow in the other anatomical structures. Furthermore, arterial blood flow typically has different characteristics from veinous blood flow. For example, arterial blood flow is typically pulsatile (e.g. characterized by variation of blood velocity within a first range according to a first periodic pattern), whereas veinous blood flow is typically characterized by variation of blood velocity within a second range, smaller than the first range, according to a second periodic pattern.

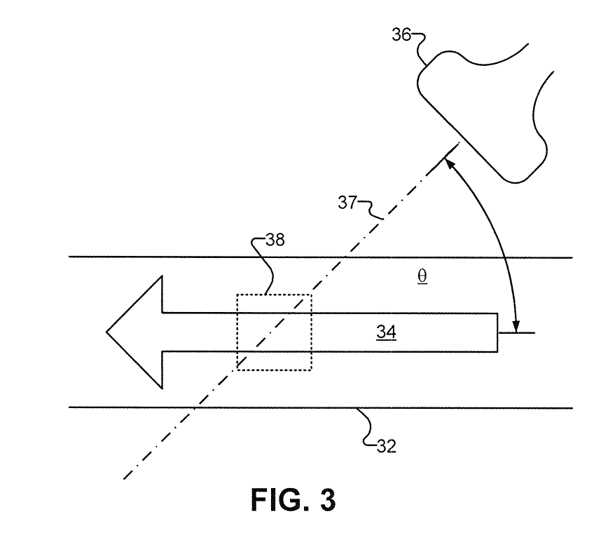

Pulse-Wave (PW) Doppler ultrasound is used in medical ultrasound exams to examine blood flow and measure blood flow velocity. FIG. 3 is a schematic depiction illustrating the application of PW Doppler ultrasound to measure blood flow velocity. A blood vessel 32 contains blood flowing in the direction indicated by arrow 34. An ultrasound probe 36 transmits a series of ultrasound pulses along a scan line 37. Pulses are reflected back to ultrasound probe 36 by structures along scan line 37. Signals reflected from a sample volume 38 at a particular depth along scan line 37 may be identified by time-gating the registration of reflected pulses received at probe 36.

Transmitted pulses incident on sample volume 38 within blood vessel 32 are reflected by blood components, such as erythrocytes, moving in the flowing blood. Because the transmitted pulses interact with moving blood components, such as erythrocytes, the frequency of the reflected pulses will differ from the frequency of the transmitted pulses. This change in frequency is known as a Doppler shift. After determining the frequency of the pulses received from sample volume 38, the velocity of blood flowing through the target volume may be calculated from the frequency shift using the Doppler equation

$$V = \frac{{F}_{D} c}{2 {f}_{0}}$$

where V is the velocity in the sample volume, F_{D} is the Doppler shift, c is the velocity of sound in blood, and f₀ is the transmitted frequency.

Because scan line 37 is at an angle to the direction of the blood flow, the Doppler shift indicates only the component of blood flow velocity parallel to scan line 37. To obtain the true blood flow velocity, the measured velocity value can be angle-corrected for the angle θ between scan line 37 and the direction of blood flow 24. This may be achieved, for example, by dividing the measured velocity value by the cosine of the angle θ between the scan line 37 and the blood flow direction 34. Estimates of the angle θ may be used to angle-correct velocity measurements. The particular estimate of the angle θ used to angle-correct measured velocity values may be specified manually (e.g., by a technician using a user interface control associated with controller 11, such as a knob, for example) or automatically according to known or future developed apparatus and/or techniques.

Various techniques for obtaining blood flow velocity measurements are known, and the reader is referred to co-owned U.S. patent application Ser. No. 13/021,676 entitled ULTRASOUND PULSE-WAVE DOPPLER MEASUREMENT OF BLOOD FLOW VELOCITY AND/OR TURBULENCE.

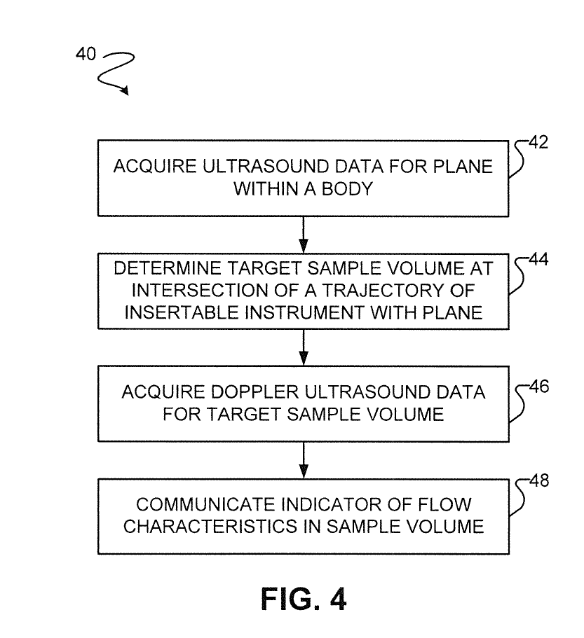

FIG. 4 is a flowchart of a method 40 according to an example embodiment for communicating an indicator of blood flow characteristics at an intersection of a trajectory of an insertable instrument and a plane in a body. Step 42 comprises acquiring ultrasound data for a plane within a body. Ultrasound data acquired in step 42 may be suitable for constructing an ultrasound image of anatomy in the plane, for example.

Step 44 comprises determining a target sample volume at the intersection of a trajectory of an insertable instrument with the plane. It will be appreciated that the intersection can be determined from the location and orientation of the insertable instrument and the location of the plane. For example, step 44 may comprise determining a target sample volume at the intersection of the trajectory 21A of needle 21 and region 26. Where the spatial relationship between trajectory 21A of needle 21 and region 26 are known (e.g., because it has been determined based on information sensed by position base station 17), controller 11 may, in performing step 44, determine the target sample volume to be sample volume 28.

Various techniques are known for determining a location of an intersection between a trajectory of an insertable instrument and the location of a region in a body for which ultrasound data is acquired, and the reader is referred to co-owned PCT Patent Application serial no. PCT/CA2010/000740, entitled FREEHAND ULTRASOUND IMAGING SYSTEMS AND ASSOCIATED METHODS.

Step 46 comprises acquiring Doppler ultrasound data for the target sample volume determined in step 44. Step 46 may comprise, for example, time-gating received pulses to sample pulses corresponding to reflections originating in the location of the target sample volume, as described in co-owned U.S. patent application Ser. No. 13/021,676, entitled ULTRASOUND PULSE-WAVE DOPPLER MEASUREMENT OF BLOOD FLOW VELOCITY AND/OR TURBULENCE.

Step 48 comprises communicating an indicator of flow characteristics in the sample volume, which indicator is based on the acquired Doppler ultrasound data for the target sample volume. Communicating an indicator of flow characteristics in the sample volume in step 48 may comprise one or more of the following, for example:
presenting a Doppler shift determined from the Doppler ultrasound data audibly;
displaying a time-velocity Doppler ultrasound spectrum determined from the Doppler ultrasound data visually;
displaying a symbolic indicator (e.g., a number, character, symbol, combination thereof, or the like) of a flow characteristic determined from the Doppler ultrasound data visually; and
the like.

The indicator communicated in step 48 may be used by a person performing a medical procedure to determine whether continued insertion of the insertable instrument along its current trajectory will cause the instrument to penetrate an artery or vein. Where a user is free to alter the trajectory of needle 21 (for example where needle 21 comprises a free-hand needle or where needle 21 is supported in an adjustable guide) the position of sample volume 28 in the image may be automatically updated in response to changes in the trajectory.

Where an ultrasound probe is used in step 42 to acquire ultrasound data for a plane in the body, and one or both of the ultrasound probe and the insertable instrument is handheld or hand-manipulable (e.g., where needle 21 is supported in an adjustable guide), it may occur that the intersection of the trajectory and the plane moves due to unsteadiness of the hand(s) holding the probe and/or instrument. Where the target sample volume is determined by the instantaneous location of the intersection, the corresponding unsteadiness of the target sample volume may interfere with or prevent the acquisition of Doppler ultrasound data in step 46. In some embodiments, the target sample volume is determined such that it is relatively more steady than the instantaneous location of the intersection. This may be done by, for example:
fixing the target sample volume at a location based one or more present and/or historical locations of the intersection;
continually (or continuously, depending on implementation) determining the target sample volume based on two or more present and/or historical locations of the intersection; and
the like.

In some embodiments, the target sample volume is fixed relative to one of: the receiver used in acquiring ultrasound data for the plane (e.g., ultrasound probe 12), the receiver used in acquiring Doppler ultrasound data for the target volume (which may be the same as or co-located with the receiver used in acquiring ultrasound data for the plane), an immobile reference (e.g., position base unit 17), or a marker secured to the body (e.g., on skin in the vicinity of the plane), for example. In some embodiments, when the target sample volume is fixed, the time-gating of received PW Doppler pulses does not change when the trajectory of the insertable instrument moves relative to the receiver used in acquiring ultrasound data for the plane.

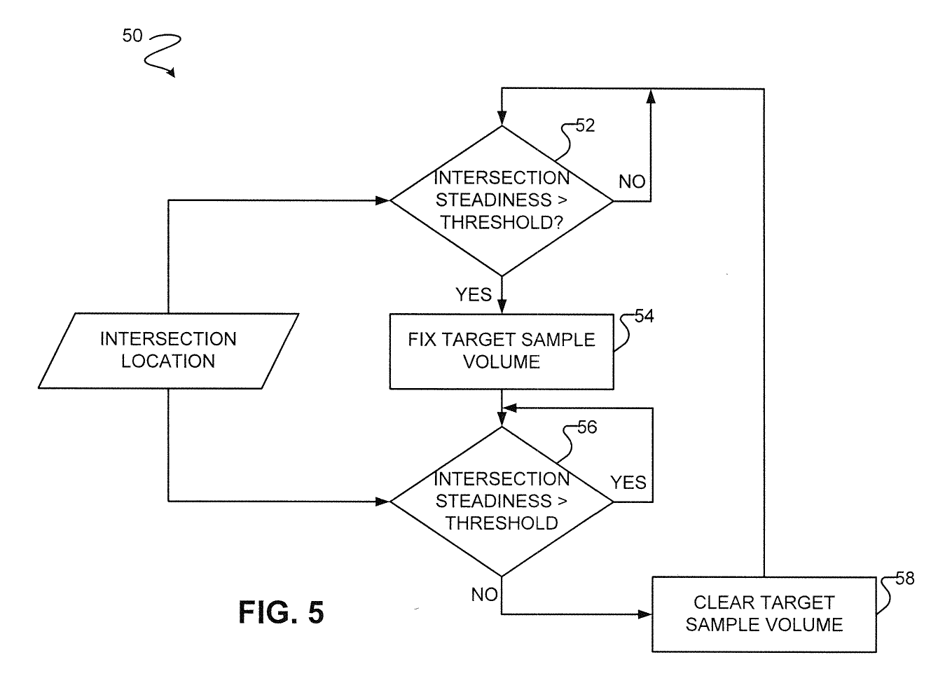

FIG. 5 shows a flowchart of an example method 50 for automatically fixing a target sample volume. Step 52 comprises determining whether the intersection of a trajectory of an insertable instrument with a plane has greater than a first threshold steadiness. The steadiness of the intersection may be determined based on various indicators of movement of the intersection, such as:
the maximum distance between any two locations of the intersection over a pre-determined time period;
the average velocity of the intersection (e.g., relative to the plane) over a pre-determined time period;
the radius of the smallest circle that encloses all locations of the intersection over a pre-determined time period; and
the like.
   The time period over which steadiness is measured may comprise a continuously moving time-window (e.g,. a time-window extending to the present).

If in step 52 it is determined that the intersection has greater than the first threshold steadiness (step 52 YES), method 50 proceeds to step 54. If in step 52 it is determined that the intersection does not have greater than the first threshold steadiness (step 52 NO), method 50 remains in step 52. Step 52 may be repeated continuously while the intersection does not have greater than the first threshold steadiness.

In step 54, the target sample volume is fixed. Step 54 may comprise fixing the sample volume at the location of the intersection when step 54 is entered, at an average of the location of the intersection over the pre-determined time period during which at least threshold steadiness was observed in step 52, at a location determined by some other function of the current and/or past locations of the intersection, or the like, for example.

After step 54, method 50 proceeds to step 56. Step 56 comprises determining whether the intersection has greater than a second threshold steadiness. Steadiness in step 56 may be determined in the same manner as in step 52, or in a different manner. For instance, steadiness in step 56 may be determined based on the distance between the current location of the intersection and the location of the sample volume fixed in step 54. If in step 56 it is determined that the intersection has greater than the second threshold steadiness (step 56 YES), method 50 remains in step 56 and the target sample volume is unchanged. Step 56 may be repeated continuously while the intersection has greater than the second threshold steadiness. If in step 56 it is determined that the intersection does not have greater than the first threshold steadiness (step 56 NO), method 50 proceeds to step 58. In step 58, the target sample volume is cleared. After step 56, method 50 returns to step 52.

In method 50, the first threshold steadiness and second threshold steadiness may be the same or different. In some embodiments, the second threshold steadiness is less than the first threshold steadiness (e.g., fixing the sample volume requires a relatively more steady intersection, but once fixed the sample volume may be maintained with a relatively less steady intersection).

In some embodiments, the target sample volume is not fixed in step 54 but instead determined based on two or more present and/or historical locations of the intersection (e.g., as an average of intersection locations) and is continually (or continuously, depending on implementation) determined in step 56 based on two or more present and/or historical locations of the intersection (e.g., as a moving window average of intersection locations). In such embodiments, steadiness in step 56 may be determined based on the distance between the current location of the intersection and the determined location of the sample volume, the distance between the current location of the intersection and one or more previous locations of the intersection (e.g., the next most recent location of the intersection), or the like, for example.

Method 40 may comprise method 50. For example, step 44 of method 40 may comprise method steps 52 and 54 (e.g., the target sample volume determined in step 44 may comprise a sample volume fixed in step 54 of method 50) and steps 46 and 48 of method 40 may be performed while method 50 remains in step 56 (e.g., Doppler ultrasound data may be acquired for the target sample volume and an indicator of flow characteristics based thereon communicated while the sample volume remains fixed).

In some embodiments, step 44 of method 40 comprises determining the target sample volume in response to user input. For example, determining the target sample volume in step 44 may comprise fixing the target sample volume at the current location of the intersection in response to a user input (e.g., a button push). Similarly, a user input may trigger a target sample volume acquisition mode in which the target sample volume is continually (or continuously, depending on implementation) determined based on two or more present and/or historical locations of the intersection. The target sample volume may be subsequently de-selected (or target sample volume acquisition mode exited) manually (e.g., by another button push) or automatically (e.g., when less than a threshold steadiness is observed, when the intersection is moved more than a threshold distance from the determined target sample volume, after a pre-determined time period has elapsed, etc.).

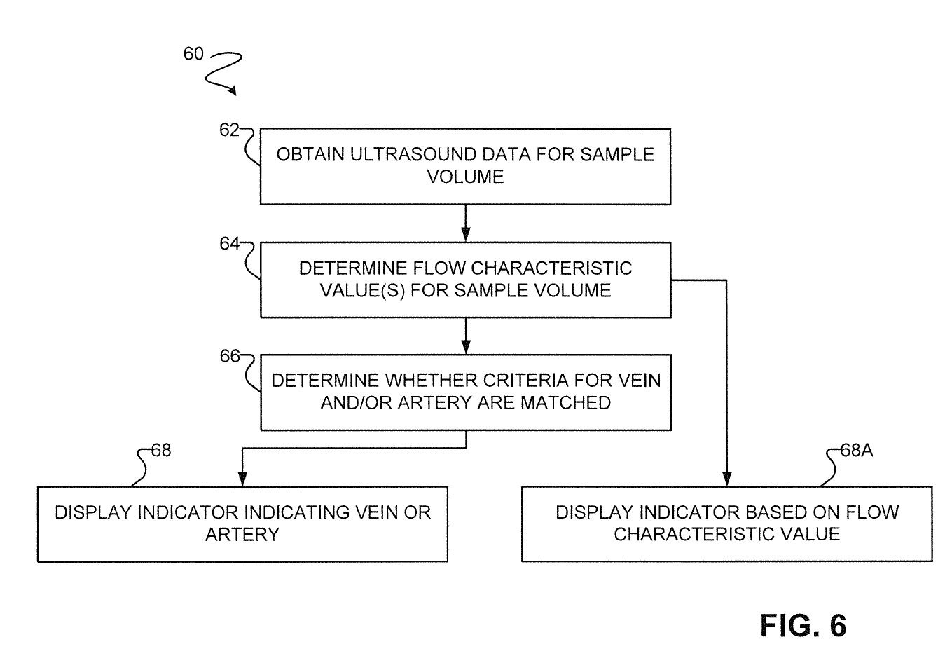

Some embodiments provide methods which automatically determine whether Doppler ultrasound data for a target sample volume is consistent for one or more of an artery and a vein, An example of such a method is method 60, a flowchart of which is shown in FIG. 6. Steps 46 and 48 of method 40 may comprise all or part of method 60.

In step 62, Doppler ultrasound data for a sample volume is obtained. In step 64, one or more flow characteristic values for the sample volume are determined based on the ultrasound data acquired in step 62. Step 64 may comprise determining a velocity magnitude value based on Doppler ultrasound data, for example. Such a value may be indicative of instantaneous velocity magnitude or be a time-series statistic derived from a plurality of instantaneous a velocity magnitude values (e.g., a statistic indicative of the central tendency, variability of velocity magnitude, or the like). Non-limiting examples of statistics that may be computed in step 64 include:
mean velocity magnitude,
median velocity magnitude,
standard deviation of velocity magnitude,
peak velocity magnitude,
minimum velocity magnitude,
velocity magnitude range,
mean velocity magnitude absolute slope (rate of change),
maximum velocity magnitude absolute slope (rate of change),
mean time between consecutive local velocity magnitude maxima,
mean time between consecutive local velocity magnitude minima,
mean time between consecutive local velocity maxima and minima,
mean time between consecutive local velocity minima and maxima, and
the like.

The above statistics may be determined on a moving time-window basis, for example. Where velocity magnitude determined in step 64 varies periodically, such a moving time-window may be synchronized to the period of such variations. In some embodiments, one or more of the above statistics is determined on a time-weighted basis (e.g., more recent samples being assigned greater weight).

In some embodiments, a flow characteristic value determined in step 64 comprises a measure of pulsatility of flow, such as pulsatility index, or measure of resistivity of flow, such as resistivity index. Pulsatility index quantifies the pulsatility or oscillations of a blood velocity waveform. Various definitions of pulsatility index are known. An example definition of pulsatility index (PI) that may be used in some embodiments is

$$P I - \frac{{V}_{max} - {V}_{min}}{{V}_{max mean}}$$

where Vₘₐₓ is the peak systolic velocity, Vₘᵢₙ is the minimum forward diastolic velocity in unidirectional flow, or the maximum negative velocity in diastolic flow reversal, and V_{max mean} is the maximum velocity averaged over at least one cardiac cycle.

Resistivity index quantifies resistance to blood flow in a blood vessel. An example definition of resistivity index (RI) that may be used in some embodiments is

$$R I = \frac{{V}_{max}}{{V}_{max} - {V}_{min}}$$

where Vₘₐₓ is the peak systolic velocity and Vₘᵢₙ is the minimum forward diastolic velocity in unidirectional flow. Pulsatility index and resistivity index may be estimated from a time-velocity spectral display of Doppler ultrasound. Where a flow characteristic value determined in step 64 comprises or is based on pulsatility index and/or resistivity, values for Vₘₐₓ, Vₘᵢₙ, and V_{max mean} may be determined from a trace of the envelope of the Doppler spectrum (e.g., step 44 may comprise tracing the Doppler spectrum and determining values for Vₘₐₓ, Vₘᵢₙ, and Vₘₐₓ mean from the trace).

In step 66, a vessel identification criterion is applied to the Doppler ultrasound data and/or to one or more values determined in step 64. The vessel identification criterion determines whether or not the Doppler ultrasound data and/or statistics match an arterial pattern or a veinous blood pattern.

In step 68, an indicator whose appearance is based on whether or not the vessel identification criterion is matched in step 66 is displayed. The indicator may be displayed on the same display used to display an ultrasound image of the body to which the sample volume belongs, for example. In some embodiments, the indicator merely indicates that the target corresponds to a vein or corresponds to an artery. In addition or in the alternative, in step 68A an indicator may be displayed that is based directly on the Doppler ultrasound data and/or one or more values derived from of the Doppler ultrasound data. Because arterial blood flow and veinous blood flow have different flow characteristics, a user viewing such an indicator may be able to determine whether the sample volume is consistent with being in one of an artery and a vein based on the appearance of the indicator.

For instance, step 68A may comprise displaying an indicator whose appearance (e.g., color, size, brightness and/or the like) is coded according to a flow characteristic value determined in step 64. In some embodiments, step 68A comprises displaying an indicator whose appearance is coded according to instantaneous velocity magnitude determined in step 64. Since instantaneous velocity magnitude for a sample volume in an artery will vary according to pattern different than the pattern according to which velocity magnitude for a sample volume in a vein varies, a user viewing the indicator may be able to determine whether the sample volume is consistent with being in one of an artery and a vein based on the changing appearance of the indicator. For example, where an indicator's brightness is coded according to instantaneous velocity magnitude (e.g., such that the indicator is displayed relatively brighter for higher velocity magnitudes than for lower velocity magnitudes), the indicator will pulse more brightly and more distinctly (i.e. having greater difference between minimum and maximum brightnesses) when the sample volume is located in an artery than when the sample volume is located in a vein.

In some embodiments, step 66 comprises determining whether determined flow characteristic value(s) for the sample volume are consistent with one or more of an artery, a vein and/or neither an artery and a vein and step 68 comprises displaying an indication of whether the determined flow characteristic value(s) for the sample volume are consistent with the one of the one or more of an artery, a vein and/or neither an artery and a vein. Step 68 may comprise, for example, displaying one or more textual and/or graphical elements indicative of determined consistency (or lack of consistency) of a determined flow characteristic value with the one of the one or more of an artery, a vein, and neither an artery nor a vein. A user viewing such an indication may use the indication to determine the location of a sample volume having flow characteristics consistent with one of an artery, a vein and/or neither an artery and a vein, and/or to confirm that a particular sample volume is within an artery or vein, for example.

In some embodiments, display of textual and/or graphical elements is contingent on whether determined flow characteristic(s) for the sample volume are determined to be consistent one of an artery and a vein. For example, step 68 may comprise displaying a first marker at an image location corresponding to the intersection of the trajectory of an insertable instrument with the region depicted in the image when the sample volume at the intersection has a flow characteristic value that is consistent with an artery, displaying a second marker different from the first marker at the image location when the sample volume has a flow characteristic value consistent with a vein, and displaying no marker (or, alternatively, a third marker different from the first and second markers) at the image location when the sample volume has a flow characteristic value not consistent with either of an artery or a vein.

In some embodiments, step 68 and/or step 68A comprises displaying on an ultrasound image an indicator of the location of the sample volume of the a portion of the body to which the sample volume belongs (e.g., a portion that includes the sample volume or a portion that does not include the sample volume). The appearance of such an indicator may be based on the flow characteristic value(s) determined in step 64 or the result of matching in step 66 (e.g., the indicator of the location of the sample volume may be the same as the indicator based on the flow characteristic value(s) determined in step 64 and/or the indicator indicating whether or not the vessel identification criterion matched in step 66).

In some embodiments, ultrasound system 10 is pre-configured to prompt a user to locate a specific artery. The procedure may lead the user step-by-step to locate the artery. In such embodiments, the first marker may invite the user to proceed (e.g., the first marker may comprise a green circle, a check mark or the like). The second marker may indicate that the user should not proceed (e.g., the second marker may comprise an X, a flashing ‘NO’, or the like). Other procedures may lead the user to locate veins. In such embodiments, a user may run the procedure. The ultrasound system may optionally display an image indicating approximately where a probe and needle should be positioned relative to a patient's anatomy. The user can then place the probe to acquire ultrasound data until an image including the desired artery is seen. The user can then manipulate the needle until a displayed trajectory indicates that the needle trajectory will intersect the artery. The system automatically performs Doppler ultrasound at the location of the intersection of the trajectory and image plane and determines if the Doppler ultrasound data indicates an artery. If so, an indicator is operated to indicate that the user may proceed. The user may than insert needle 21 or another implement until its tip penetrates the artery. The user may monitor the progress of the needle 21 on a display of the ultrasound system. In some embodiments, an indicator is operated to indicate that the tip of needle 21 has penetrated the artery. When the tip of needle 21 enters the artery the user may, for example, withdraw fluid from the artery or inject fluid into the artery by way of needle 21.

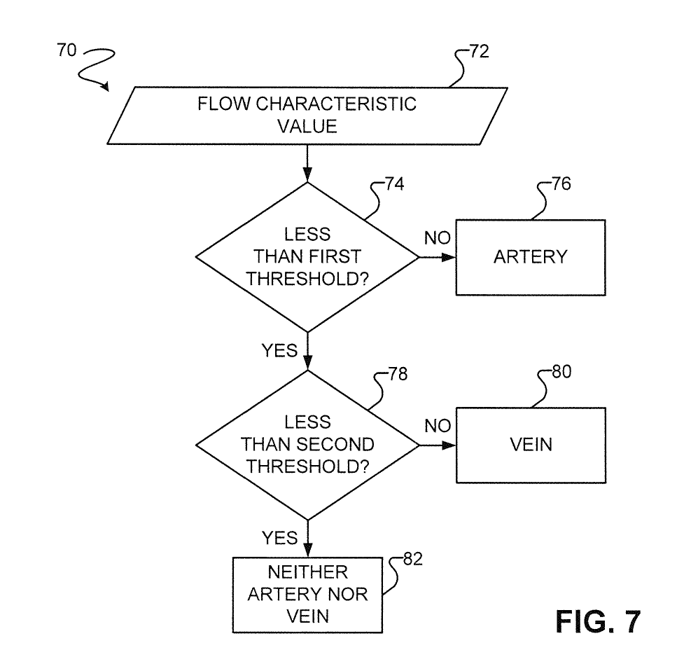

In some embodiments, step 66 comprises comparing at least one of the one or more determined flow characteristic values of the sample volume with a threshold, determining whether the comparison indicates consistency with one the one or more of an artery, a vein and/or neither an artery and a vein, and displaying an indication of the determined result. Step 66 may, for example, comprise method 70 shown in FIG. 6. Method 70 applies the fact, illustrated by diagram 90 in FIG. 7, that typical values of certain flow characteristics for arteries, veins and anatomical structures that are neither arteries nor veins fall within ordered, non-overlapping ranges (in diagram 90, ranges 92, 94 and 96, respectively). Method 70 compares a value 72 for such a flow characteristic to thresholds between the adjacent endpoints of the ranges of typical values for the flow characteristic (in diagram 90, first threshold 98 between adjacent endpoints of ranges 92 and 94, and second threshold 99 between adjacent endpoints of ranges 94 and 96) in order to determine whether the value 72 is consistent with an artery, a vein or an anatomical structure that is neither an artery nor a vein. Flow characteristic value 72 may comprise a value indicative of peak velocity magnitude, central tendency of velocity magnitude (e.g., mean, median, or the like), variability of velocity magnitude (e.g., range, standard deviation, variance, or the like), pulsatility, or the like, for example.

In step 74 of method 70, flow characteristic value 72 is compared with a first threshold. If in step 74 it is determined that flow characteristic value 72 is not less than the first threshold (step 74 NO), then it is determined that flow characteristic value 72 is consistent with an artery (step 76). If in step 74 it is determined that flow characteristic value 72 is less than the first threshold (step 74 YES), then method 70 proceeds to step 78.

In step 78 of method 70, flow characteristic value 72 is compared with a second threshold less than the first threshold. If in step 78 it is determined that flow characteristic value 72 is not less than the second threshold (step 78 NO), then it is determined that flow characteristic value 72 is consistent with a vein (step 80). If in step 78 it is determined that flow characteristic value 72 is less than the second threshold (step 78 YES), then it is determined that flow characteristic value 72 is consistent with an anatomical structure that is neither an artery nor a vein (step 82).

Components of system 10 may be configured to perform all or part of methods 40, 50, 60 and 70. For example, controller 11 may be configured to do one or more of the following:
in conjunction with probe 12, acquire ultrasound data for a plane within the body of patient P (e.g., in performing step 42);
determine a target sample volume at the intersection of the trajectory 21A of needle 21 based on position information for probe 12 and/or needle 21, which information is sensed by position base station 17 (e.g., in performing step 44);
determine whether the intersection of the trajectory 21A of needle 21 with a plane for which ultrasound data is acquired by probe 12 has or does not have at least threshold steadiness based on position information for probe 12 and/or needle 21, which information is sensed by position base station 17 (e.g., in performing step 44, such as by performing method 50);
in conjunction with probe 12, acquire Doppler ultrasound data for the target sample volume(s) (e.g., in performing step 46);
in conjunction with display 13 and/or headphones 16, communicate an indicator of flow characteristics in the target sample volume (e.g., in performing step 48);
determine one or more flow characteristic values for the target sample volume(s) based on the acquired Doppler ultrasound data (e.g., in performing step 64);
cause display 13 to display an indicator whose appearance is based on determined flow characteristic value(s) (e.g., in performing step 68A);
determine whether determined flow characteristic value(s) are consistent with one or more of an artery, a vein and/or neither an artery and a vein (e.g., in performing step 66, such as by performing method 70); and
cause display 13 to display an indication of whether the determined flow characteristic value(s) are consistent with the one of the one or more of an artery, a vein and/or neither an artery and a vein (e.g., in performing step 68).

Where a component (e.g. a controller, display, audio monitor, user interface, probe, instrument, position base station, position sensor, etc.) is referred to above, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

Aspects of the invention may be provided in the form of a program product. The program product may comprise any medium which carries a set of computer-readable information comprising instructions which, when executed by a data processor, cause the data processor to execute a method of the invention. Program products according to the invention may be in any of a wide variety of forms. The program product may comprise, for example, physical media such as magnetic data storage media including floppy diskettes, hard disk drives, optical data storage media including CD ROMs, DVDs, electronic data storage media including ROMs, flash RAM, or the like. The computer-readable information on the program product may optionally be compressed or encrypted.

Those skilled in the art will appreciate that certain features of embodiments described herein may be used in combination with features of other embodiments described herein, and that embodiments described herein may be practised or implemented without all of the features ascribed to them herein. Such variations on described embodiments that would be apparent to the skilled addressee, including variations comprising mixing and matching of features from different embodiments, are within the scope of this invention.

It will be appreciated that the invention may be embodied in a wide variety of embodiments. For example, embodiments of the invention may comprise:
methods for identifying the locations of blood vessels;
ultrasound imaging methods;
ultrasound apparatus;
devices for processing ultrasound data to identify seeds or other implantable items;
computer media carrying instructions that when executed cause computers to perform methods for identifying locations of blood vessels;
computer media carrying instructions to perform ultrasound imaging methods;
etc.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

What is claimed is:

1. A method for targeting a blood vessel, the method comprising:
acquiring ultrasound data for a plane within a body;
automatically determining a target sample volume at an intersection of a trajectory of an insertable instrument with the plane;
acquiring Doppler ultrasound data at least for the target sample volume at the intersection; and
communicating an indicator of flow characteristics in the target sample volume, the indicator based on the Doppler ultrasound data.

2. The method of claim 1 wherein communicating the indicator of flow characteristics in the target sample volume comprises presenting a Doppler shift determined from the Doppler ultrasound data audibly.

3. The method of claim 1 wherein communicating the indicator of flow characteristics in the target sample volume comprises displaying a time-velocity Doppler ultrasound spectrum determined from the Doppler ultrasound data.

4. The method of claim 1 comprising:
determining a flow characteristic value based on the Doppler ultrasound data, and
wherein communicating the indicator of flow characteristics in the target sample volume comprises displaying the flow characteristic value.

5. The method of claim 4 wherein the flow characteristic value comprises a measure of pulsatility of flow at the target sample volume.

6. The method of claim 4 wherein the flow characteristic value comprises a measure of resistivity of flow at the target sample volume.

7. The method of claim 4 comprising:
tracing an envelope of a time-velocity Doppler ultrasound spectrum determined from the Doppler ultrasound data, and
wherein determining the flow characteristic value comprises determining the flow characteristic value based on velocity information obtained from the trace of the envelope of the time-velocity Doppler ultrasound spectrum.

8. The method of claim 4 wherein the flow characteristic value comprises a velocity magnitude value and wherein an appearance of the indicator is coded according to the velocity magnitude value.

9. The method of claim 8 wherein one or more of the brightness of the indicator, the color of the indicator, the size of the indicator, and the shape of the indicator is coded according to the velocity magnitude value.

10. The method of claim 8 wherein the velocity magnitude value comprises an instantaneous velocity magnitude.

11. The method of claim 8 wherein the velocity magnitude value comprises a central tendency statistic of velocity magnitude.

12. The method of claim 8 wherein the velocity magnitude value comprises a variability statistic of velocity magnitude.

13. The method of claim 1 comprising:
determining whether the determined flow characteristic value is consistent with one of an artery and a vein,
wherein the indicator based on the flow characteristic value comprises an indicator indicative of whether the determined flow characteristic value is consistent with the one of an artery and a vein.

14. The method of claim 13 wherein determining whether the determined flow characteristic value is consistent with one of an artery and a vein comprises comparing the determined flow characteristic value with a first threshold.

15. The method of claim 13 wherein determining whether the determined flow characteristic value is consistent with one of an artery and a vein comprises determining that the determined flow characteristic value is consistent with an artery when the determined flow characteristic value is greater than the first threshold.

16. The method of claim 14 wherein determining whether the determined flow characteristic value is consistent with the one of an artery and a vein comprises determining that the determined flow characteristic value is consistent with a vein when the determined flow characteristic value is less than the first threshold.

17. The method of claim 1 comprising:
comparing the determined flow characteristic value with a second threshold, the second threshold less than the first threshold; and
determining that the determined flow characteristic value is consistent with neither one of an artery and a vein when the determined flow characteristic value is less than the second threshold.

18. The method of claim 1 comprising acquiring ultrasound image data using the ultrasound probe and generating an ultrasound image based on the acquired ultrasound image data.

19. The method of claim 18 comprising displaying the ultrasound image on a display and wherein displaying an indicator based on the flow characteristic value comprises displaying the indicator on the display.

20. The method of claim 19 wherein displaying the indicator on the display comprises displaying the indicator adjacent the image.

21. The method of claim 19 wherein displaying the indicator on the display comprises superposing the indicator on the image.

22. The method of claim 19 wherein displaying the indicator on the display comprises superposing the indicator on the image at an image location indicative of the location of the target sample volume relative to the portion of the body depicted in the image.

23. The method of claim 1 comprising displaying an indication of the location of the target sample volume.

24. The method of claim 23 wherein displaying an indicator based on the flow characteristic value comprises displaying an indication of the location of the target sample volume.

25. The method of claim 1 wherein automatically determining the target sample volume at the intersection of the trajectory of the insertable instrument with the plane comprises fixing the target sample volume at a current location of the intersection of the trajectory with the plane.

26. The method of claim 1 wherein automatically determining the target sample volume at the intersection of the trajectory of the insertable instrument with the plane comprises determining the target sample volume based on at least one historical location of the intersection of the trajectory with the plane.

27. The method of claim 1 comprising:
monitoring a steadiness of the intersection of the trajectory of the insertable instrument with the plane; and
automatically determining the target sample volume when at least a threshold steadiness is observed.

28. The method of claim 1 comprising automatically determining the target sample volume in response to a user input.

29. An ultrasound system for use in targeting a blood vessel with an insertable instrument, the ultrasound system comprising:
an ultrasound transducer operable to receive ultrasound echo signals returning from a portion of the body;
a position sensing system operable to monitor a spatial location and orientation of the instrument and a spatial location and orientation of the ultrasound transducer;
a controller communicatively coupled to the ultrasound transducer and the position sensing system; and
a user interface communicatively coupled to the controller;
wherein the controller is configured to:
cause the ultrasound transducer to acquire ultrasound data for a plane within a body;
determine a target sample volume at an intersection of a trajectory of the insertable instrument with the plane;
cause the ultrasound transducer to acquire Doppler ultrasound data at least for the target sample volume at the intersection; and
communicate an indicator of flow characteristics in the target sample volume via the user interface, the indicator based on the Doppler ultrasound data.

30. The system of claim 29 wherein the user interface comprises an audio monitor, and the indicator of flow characteristics in the target sample volume comprises an audible presentation of a Doppler shift determined from the Doppler ultrasound data.

31. The system of claim 29 wherein the user interface comprises a display, and the indicator of flow characteristics in the target sample volume comprises a time-velocity Doppler ultrasound spectrum determined from the Doppler ultrasound data.

32. The system of claim 29 wherein the controller is configured to:
determine a flow characteristic value based on the Doppler ultrasound data, and
wherein the indicator of flow characteristics in the target sample volume comprises a the flow characteristic value.

33. The system of claim 32 wherein the flow characteristic value comprises a measure of pulsatility of flow at the target sample volume.

34. The system of claim 32 wherein the flow characteristic value comprises a measure of resistivity of flow at the target sample volume.

35. The system of claim 32 wherein the controller is configured to:
trace an envelope of a time-velocity Doppler ultrasound spectrum determined from the Doppler ultrasound data, and
determine the flow characteristic value based on velocity information obtained from the trace of the envelope of the time-velocity Doppler ultrasound spectrum.

36. The system of claim 32 wherein the flow characteristic value comprises a velocity magnitude value and wherein an appearance of the indicator is coded according to the velocity magnitude value.

37. The system of claim 36 wherein one or more of the brightness of the indicator, the color of the indicator, the size of the indicator, and the shape of the indicator is coded according to the velocity magnitude value.

38. The system of claim 36 wherein the velocity magnitude value comprises an instantaneous velocity magnitude.

39. The system of claim 36 wherein the velocity magnitude value comprises a central tendency statistic of velocity magnitude.

40. The system of claim 36 wherein the velocity magnitude value comprises a variability statistic of velocity magnitude.

41. The system of claim 29 wherein the controller is configured to:
determine whether the determined flow characteristic value is consistent with one of an artery and a vein,
wherein the indicator based on the flow characteristic value comprises an indicator indicative of whether the determined flow characteristic value is consistent with the one of an artery and a vein.

42. The system of claim 41 wherein the controller is configured to:
determine whether the determined flow characteristic value is consistent with one of an artery and a vein by at least comparing the determined flow characteristic value with a first threshold.

43. The system of claim 41 wherein the controller is configured to:
determine that the determined flow characteristic value is consistent with an artery when the determined flow characteristic value is greater than the first threshold.

44. The system of claim 42 wherein the controller is configured to:
determine that the determined flow characteristic value is consistent with a vein when the determined flow characteristic value is less than the first threshold.

45. The system of claim 29 wherein the controller is configured to:
compare the determined flow characteristic value with a second threshold, the second threshold less than the first threshold; and
determine that the determined flow characteristic value is consistent with neither one of an artery and a vein when the determined flow characteristic value is less than the second threshold.

46. The system of claim 31 wherein the controller is configured to:
cause the ultrasound probe to acquire ultrasound image data;
generate an ultrasound image based on the acquired ultrasound image data; and
display the ultrasound image on the display.

47. The system of claim 46 wherein the controller is configured to display the indicator of flow characteristics in the target sample volume on the display.

48. The system of claim 47 wherein the controller is configured to display the indicator adjacent the image.

49. The system of claim 47 wherein the controller is configured to superpose the indicator on the image.

50. The system of claim 47 wherein the controller is configured to superpose the indicator on the image at an image location indicative of the location of the target sample volume relative to the portion of the body depicted in the image.

51. The system of claim 29 wherein the controller is configured to display an indication of the location of the target sample volume.

52. The system of claim 29 wherein the controller is configured to automatically determine the target sample volume at the intersection of the trajectory of the insertable instrument with the plane by fixing the target sample volume at a current location of the intersection of the trajectory with the plane.

53. The system of claim 29 wherein the controller is configured to automatically determine the target sample volume at the intersection of the trajectory of the insertable instrument with the plane based on at least one historical location of the intersection of the trajectory with the plane.

54. The system of claim 29 wherein the controller is configured to:
monitor a steadiness of the intersection of the trajectory of the insertable instrument with the plane, and
automatically determine the target sample volume when at least a threshold steadiness is observed.

55. The system of claim 29 comprising:
user input apparatus,
wherein the controller is configured to automatically determine the target sample volume in response to a user input registered by the user input apparatus.

56. A method for targeting a blood vessel, the method comprising:
acquiring ultrasound data for a plane within a body;
automatically determining a location of an intersection of a trajectory of an insertable instrument with the plane;
automatically selecting a target sample volume at the location of the intersection; and
acquiring Doppler ultrasound data at least for the target sample volume at the intersection.
